(19) United States
(12) Patent Application Publication
(10) Pub. No.: US 2009/0217743 A1
(43) Pub. Date: Sep. 3, 2009

(54) DEVICE AND PROCESS FOR DETERMINING AN ORGANIC AND INORGANIC FRACTION OF A SAMPLE

(76) Inventors: Wolfgang Falkenberg, Seefeld (DE); Markus Mornhinweg, Diessen (DE)

Correspondence Address: WESTMAN CHAMPLIN & KELLY, P.A., SUITE 1400, 900 SECOND AVENUE SOUTH, MINNEAPOLIS, MN, 55402, US

(21) Appl. No.: 12/090,705

(22) PCT Filed: Oct. 23, 2006
(86) PCT No.: PCT/EP2006/010205

§ 371 Date: Aug. 12, 2008

(30) Foreign Application Priority Data
Oct. 21, 2005 (DE) 10 2005 050 601.1
Nov. 14, 2005 (EP) 05024823.6

(51) Int. Cl. G01N 5/04; G01N 1/44; G01N 1/24; G01N 33/00

(52) U.S. Cl. 73/61.72; 73/863.11; 73/863.23; 73/864.34

(57) ABSTRACT

A device is displayed for determining an organic and inorganic fraction of a sample, which comprises a suspension medium, particularly water, and also solids. A preferably tightly sealable treatment chamber is provided, which comprises a sampling device, a filter unit, which is constructed and attached to the sampling device such that the suspension medium can be removed from the sample through the filter unit, a suction device for removing the suspension medium, a heat energy source for supplying heat energy to the sample, a temperature measuring unit for recording the temperature of the sample, a weighing unit for weighing the sample and a control and data evaluation unit, for controlling the energy source and for receiving and storing output signals from the temperature measuring unit and also the weighing unit. All parts of the device are constructed so that a sample can be analysed within the treatment chamber, without having to remove it in the interim. This represents a considerable time saving.

The invention relates to a device and also a process for determining an organic and inorganic fraction of a sample and also an application thereof.

In industrial procedures, for example in paper manufacture or in the removal of waste slurries, samples must be taken continuously and as frequently as possible during the procedure, in order to be able to control and adjust the procedure according to the analysis result. Thus, for example in paper manufacture, it must be ascertained which organic fibre fractions and which inorganic pigment fractions are present in the sample, in order to optimally adjust the manufacturing parameters.

In analytical methods up till now this has been done stepwise with the aid of various measuring units. This operation is extraordinarily time-consuming, so that a considerable period of time elapses between taking the sample and obtaining the analysis result, which is entered as “dead time” in the operation control.

The object of the invention is to display to that effect a device and a process of the type specified at the outset, to enable rapid and accurate sample measurement.

This object is achieved by a process according to claim 1 and a device according to claim 15. Particular applications of the device and the process are described in claims 27 and 28.

An essential part of the invention is that all essential steps for treating the sample and measuring the parameters that are important here are carried out in a closed system (e.g. in a treatment chamber), so that there is no need for special handling of samples and their transportation from measuring unit to measuring unit and from treatment device to treatment device.

In detail, the device comprises a preferably tightly sealable treatment chamber for determining an organic and inorganic fraction of a sample comprising a suspension medium, particularly water, and solids. This comprises a sampling device, a filter unit that is constructed and attached to the sampling device such that the suspension medium can be removed from the sample through the filter unit, a suction device to remove the suspension medium, a heat energy source to supply energy, particularly heat radiation to the sample, a temperature measuring unit to record the temperature, a weighing unit to ascertain the weight of the sample and also a control and data evaluation unit to control the heat energy source and to record and store output signals from the temperature measuring unit and from the weighing unit.

Due to the temperature and the weight of the sample being monitored essentially continuously (as far as this happens with digital systems), the individual procedural steps can be carried out in the minimum amount of time. In particular, an automated switchover can be carried out from each step to the next, which is not the case with conventional analytical systems of this type.

The filter unit comprises a filter suitable for combustion temperatures and, if necessary, also for pyrolysis temperatures, preferably a glass fibre filter or perforated plate filter, which is preferably replaceable. After removing the suspension medium, the filter is thus used as a sample carrier in the continuing procedure. The filter unit can also be used as a support structure for (later on in the process) a combusting filter material, e.g. paper. Filters made of ceramics or other similar temperature-resistant materials with all their advantages can also be used.

Different types of heat energy sources can be provided. Microwave transmitters are particularly fast and also particularly easy to adjust, operating usually at 2.455 GHz. With this energy source, the suspension medium can be heated up directly in the case of an aqueous sample, thus offering extremely good controllability. Furthermore, secondary heating elements can also be heated up using microwaves, said elements being in direct contact with the sample or converting the microwave energy into infrared radiation. By way of example, the filter mentioned at the outset can also be fabricated from a material or contain such a material that absorbs the microwave energy due to electrical conductivity, thus heating up the sample.

Alternatively, or especially also additionally, the heat energy source can comprise an infrared radiator and particularly such infrared radiators, whose wavelength is matched to the material to be heated up or to its absorption coefficients.

The sample can be introduced into the treatment chamber in a defined amount in a sample container. Alternatively, it is possible to feed the sample in small partial amounts (drops) to the filter during the de-watering operation, which in many cases can lead to faster de-watering.

A gas supply unit is preferably provided for the supply of gas, particularly air, to the treatment chamber, the gas supply unit preferably comprising units for conditioning, particularly for heating up and/or drying the supplied gas. Thus, not only is the suspension medium drawn off from the sample, but a gas is used as an aid to remove the suspension medium as quickly as possible. The gas is supplied without direction and turbulently, so that ash forming in the sample container remains and is not blown out. Of particular advantage in this arrangement is the possibility of conveying the gas through the sample, thus achieving particularly effective and rapid drying. Conveying the gas through the sample is improved if a flocculant is added to the sample, so that the filter cake becomes “porous”. Drying can be accelerated by heating up the gas, particularly air, so that a heat supply to the sample is also ensured through the hot gas. In order to heat up the sample as quickly as possible yet also as efficiently as possible, it is also possible to introduce heaters into the sample, said heaters being constructed such that they can be heated up by a radiation source, e.g. microwaves. These heaters can furthermore be constructed such that they disperse the de-watered sample for the through-flow of gas or air and also have a supportive effect during de-watering. For example, heaters made of porous or air-permeable or gas-permeable material or made of such a structure can take up the suspension medium, without fibres/solids forming and release the suspension medium again quickly when reheated.

Furthermore, gas removal units are provided that are constructed and arranged such that gas essentially flows through the sample continuously. These gas removal units can comprise a vacuum pump. It is possible to record the temperature of the sample via the gas removal units or gas flow. In this respect, a temperature measuring unit is provided, which creates a temperature measuring signal corresponding to the temperature of the gas flowing through the sample. This represents a particularly simple and effective process of temperature measurement, if necessary, a correction factor being included for the temperature loss between the sample and the measuring sensor.

In order to ascertain the drying state of the sample, there are various possibilities that can be used individually or in combination.

In an embodiment of the invention, the drying state of the sample is ascertained in that the change in mass is observed over time during essentially continuous measurement of the sample. If the change in mass does not attain a preset value, this can be used to define the dry state. Alternatively or additionally, the drying state or the dry state of the sample can then be ascertained, if the time curve of a temperature increase in the gas flow (and thus in the sample) exceeds a predetermined measurement. It is assumed for this purpose that if the sample still contains solvent which is evaporated then the temperature of the sample essentially remains constant. The temperature only rises when the heat taken up by the sample including solvent no longer has the function of evaporating the solvent, the sample is therefore dry.

Furthermore, in another embodiment, a suction underpressure is ascertained when drawing off the suspension medium. The drying state is ascertained in relation to the lateral curve of the underpressure. Then, when the underpressure rises, it can be assumed that the “gas supply”, in other words the steam from the solvent, is finished, the solvent is thus completely evaporated.

“Drawing off” always refers to the creation of a differential pressure between “above” and “below” the sample. A pressure source can also be used for drawing off instead of a prechamber pump.

Finally, in a preferred embodiment of the invention, the drying state is ascertained through absorption by radiation, particularly microwave radiation. Particularly if the sample itself is heated up by means of microwave radiation to evaporate the solvent, the measurement of absorption of microwave radiation is also a measurement of the solvent still in the sample. If the sample is solvent-free, particularly anhydrous, then only a very small portion of the microwaves is absorbed. The time curve of microwave absorption is also of great relevance here. As long as solvent is still being evaporated, absorption is high. The latter then drops relatively rapidly, when the quantity of suspension medium (water or similar liquids that can be heated by microwaves) has completely evaporated.

When the sample is combusted or ashed in the treatment chamber, then the completeness of combustion is also measured in a preferred embodiment of the invention. Firstly, this can take place by observing the substances that arise during combustion. This involves detecting CO₂ or H₂O and also detecting smoke. Furthermore, it is possible to ascertain combustion by “optical” observation, as light is emitted during combustion. Imaging processes can also be used here, by means of which it can be ascertained whether light emissions were observable over the complete surface, from which it can be concluded that each point in the sample surface was affected by this operation.

Furthermore, the control and data evaluation unit is provided with storage units and constructed such that the weight of the sample is ascertained and recorded
in the wet state to ascertain the total sample mass,
in the dried state to ascertain the sample fraction of dry mass,
after ashing, preferably at a maximum of approximately 580° C., to ascertain the organic and inorganic fraction of the sample,
after burning out, preferably at over 580° C., to ascertain the calcium carbonate fraction.

Thus, in a single measuring operation, a plurality of parameters can be held and evaluated.

Particularly the first step can also be carried out outside the treatment chamber. The mass of the sample is thus ascertained before the latter is introduced into the treatment chamber. This can also be achieved, of course, by introducing a volumetrically determined “unit sample” into the treatment chamber.

The burning out step, in which heat and oxygen is supplied to the ash until it is completely burnt out, can also be omitted—depending on the analyses that are to be carried out. Ascertaining the mass of burnt out sample residue can also be carried out outside the treatment chamber.

The control and data evaluation unit can be constructed such that the stored output signals of temperature and weight can be evaluated over time using special values, particularly extreme values from a curve of temperature and weight. As a result, it is possible not only to automise the measuring operations via computer operations, but to shorten them. For example, when drying a sample, it is possible to extrapolate the curve when the curve of weight dependent on time has been ascertained, so that the dry weight resulting from delicate drying can be calculated without actually drying the sample in this delicate manner to completion. If sufficient data for the extrapolation of the curve are available, an increased supply of energy can be used, which then leads to the next operation, namely the combustion of organic carbon compounds. It is likewise possible to shorten the process step from this combustion step after collecting a sufficient number of measuring points via an extrapolation of the resulting curve and to allow the temperature to rise to above 560-580° C. (the conversion temperature of CaCO₃ to CaO+CO₂), so that the next process step is (rapidly) reached without having to wait a long time, as was previously the case, until each process step has definitely been completed in order to then ascertain the remaining mass by weighing.

The use of the device according to the invention and the use of the process according to the invention is particularly advantageous for determining pigment and fibre fractions in a suspension for paper manufacture. Due to the high speed and simultaneous high accuracy, it is possible to control the paper manufacturing procedure in an advantageous manner.

Furthermore, the use of the device and the process is very advantageous for determining organic and inorganic carbon fractions in industrial wastewater and slurries particularly for minimising the addition of flocculants (e.g. in mining wastewater) and for calculating waste disposal values. This produces a considerable cost saving and a lower environmental burden when disposing of the waste substances.

Advantageous features of the invention will emerge from the sub-claims and the following description of embodiments, which are shown in the accompanying drawings, in which:

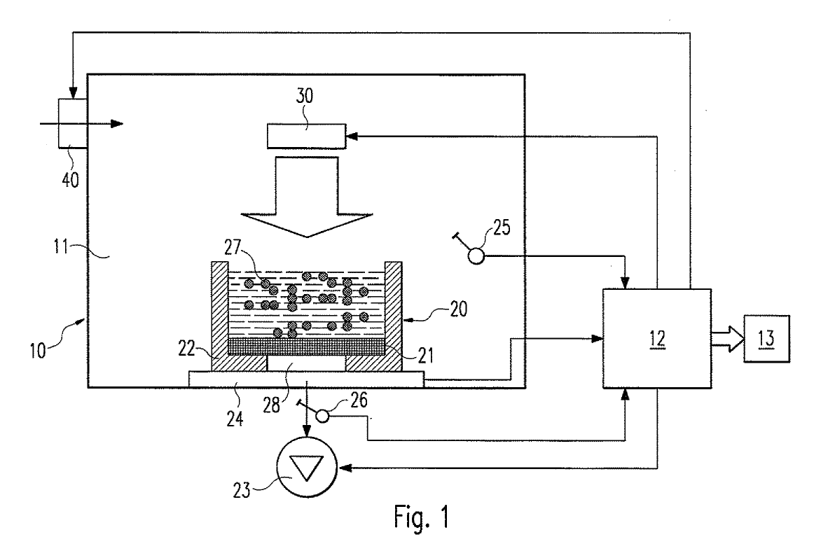

FIG. 1 is a schematic representation of a first embodiment of the invention,

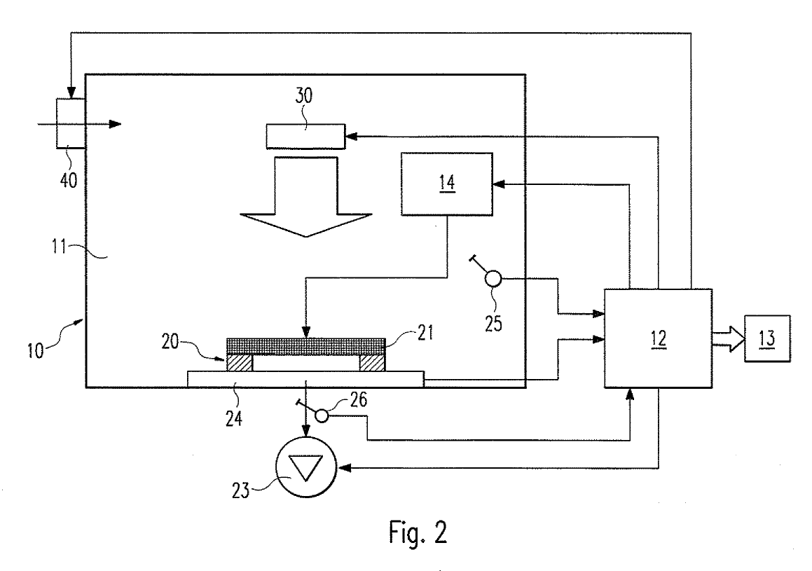

FIG. 2 is a second embodiment of the invention and

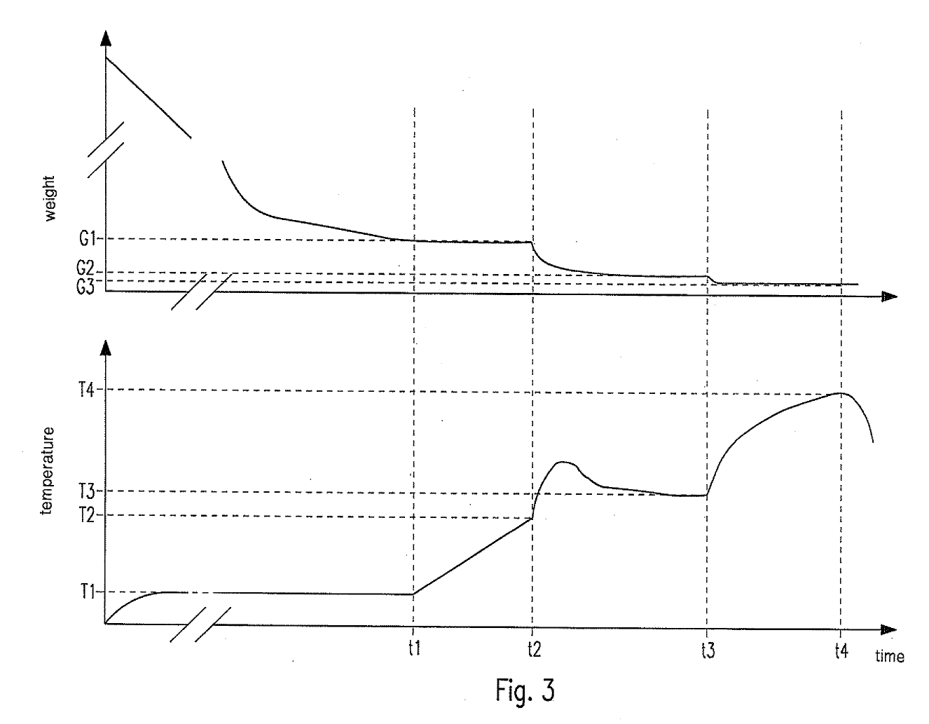

FIG. 3 is the curve of temperature and weight of a sample over time based on simple considerations to explain the process according to the invention.

In the following description the same reference numerals are used for identical and identically functioning parts.

FIG. 1 shows very schematically a treatment chamber 10, in the inner space 11 of which a sampling device 20 is located. In the embodiment shown in FIG. 1, this comprises a filter 21 arranged in a container 22, which comprises an aperture 28 under the filter 21. This aperture 28 has a gastight connection to a suction pump 22 or a vacuum pump.

The container 22, which is used for receiving the sample, is on scales 24, whose output signals represent in electrical form the weight of the sampling device 20 including contents and filter.

A heat energy source 30, which can be constructed in a known manner per se, is provided in the inner space 11. This heat energy source 30 preferably (also) comprises a microwave radiation source. An inner space temperature sensor 25 is provided to scan the temperature in the inner space 11. Furthermore, a gas flow temperature sensor 26 is provided which scans the temperature of the medium, which is drawn off by the suction pump 23.

A gas is drawn into the inner space 11 by a gas conditioning unit 40, the gas conditioning unit 40 being controlled by the control unit 12. The gas conditioning unit 40 is constructed such that the drawn in gas can be dried/preheated, so that an additional heat energy source is available. Moreover, it is possible to supply an inert gas, e.g. nitrogen, through the gas conditioning unit 40 instead of air, so that when drying a sample, even at higher temperatures, there is no danger of oxidising or combusting its constituents.

The control unit 12 also includes a data evaluation unit (a computer), with storage units for the measured signals recorded and to store ascertained results, which are reproduced on a screen 13 or a documentation unit (e.g. a plotter).

In order to be able to heat up a sample contained in the container 22 more quickly with microwave energy, it is advantageous if heaters 27 are added to the sample, said heaters being heatable by microwave energy but otherwise (as of course with the sample container 20) also behaving inertly during the complete course of the process.

The embodiment of the invention shown in FIG. 2 differs from that of FIG. 1 in that instead of the container 22, which must comprise a sufficiently large volume to take a sufficiently large sample, the sampling device 20 essentially only consists of the filter 21, under which gas is drawn off via the suction pump 23. In this case, the sample is essentially supplied continuously or dropwise through a sampling supply unit 14, which is also arranged outside the inner space 11 of the treatment chamber 10, and during the supplying is immediately de-watered. As a result, a faster de-watering can be achieved. In order to achieve such a faster de-watering in the embodiment according to FIG. 1, it is advantageous if a flocculant is added to the sample, as this leads to the sample becoming “looser” or more “gas-permeable”, whereby the de-watering operation is accelerated and the filtrate becomes clear.

Hereinafter the operation of the device according to the invention and the execution of the process according to the invention is described in more detail in FIG. 3. It should be noted at this point that this diagram reproduces a possible, not a measured, analysis curve. The temperature is that of the sample and can be measured by means of the gas flow temperature sensor 26, as the gas supplied through the sample essentially reproduces the temperature of the sample.

In a first phase the water is drawn off out of the sample. Heating is simultaneously applied, so that the temperature rises up to the temperature T1, which corresponds to the evaporation temperature of the suspension medium, that is 100° C., if it is an aqueous sample. The remainder of the water contained in the sample is evaporated under constant energy supply, the weight of the sample therefore falls until it has reached the weight G1 by time t1. At this time t1 the water (or another suspension medium) has evaporated. The temperature now rises up to time t2 with the weight G1 remaining the same.

At time t2 the sample has reached a temperature at which the organic fractions containing carbon combust. Due to the energy released here, the temperature jumps beyond the temperature T3 preset by the heat energy source (approx. 580° C.) and falls (due to the heat losses and gas/air supplied) to this temperature value T3 again. The weight of the sample continually reduces, until it has reached the value G2, which represents the weight of the sample at which the sample is combusted and ashed, whereby the fraction of inorganic and organic solids can be ascertained.

Upon heating the sample further to a temperature T4, which is above 580° C., the weight of the sample falls from the value G2 to the value G3 during the period of t3-t4, so that now the mass of calcium oxide and from it the fraction of inorganically bound carbon can be ascertained. This analysis operation is known per se.

Due to the temperature and also its weight now being monitored simultaneously via the sensors provided, it can now be ascertained over time based on the weight and/or temperature curve when each operation, in other words the de-watering/drying the combustion of organic carbons and the combustion of inorganic carbons is completed. It is thus not necessary to have safety intervals, so that the total duration of the process can be shortened.

Furthermore, it can be seen from the curves in FIG. 3 that it is possible to extrapolate the weight values produced. For example, it is thereby possible to work with increased energy supply and therefore shorter heating up times. For example, it is not necessary to wait for time t1 or even t2 in order to be able to determine the weight of the anhydrous sample, as this can already be derived from the curve of the weight loss before time t1. The same applies to the two subsequent phases, in which inorganic and organic carbon is combusted and oxidised.

A further advantage of the device according to the invention is that other suspended solids can also be rapidly and simply measured with this device. For example, pigment fractions (in other words practically without organic fractions) in a coating suspension in papermaking can be determined with this (one) apparatus, so that an “all-round measuring apparatus” is provided. The steps of combustion and pyrolysis are thereby omitted, but the suction filtering, drying and weighing are considerably accelerated.

### LIST OF REFERENCE NUMERALS

10 Treatment chamber
11 Inner space
12 Control/data evaluation unit
13 Screen/documentation
14 Sample supply unit
20 Sampling device
21 Filter
22 Container
23 Suction pump
24 Scales
25 Inner space temperature sensor
26 Gas flow temperature sensor
27 Heaters
28 Aperture
30 Heat energy source
40 Gas conditioning unit

What is claimed is:

1. Process for determining an organic and inorganic fraction of a sample containing a suspension medium and solids, comprising the following steps
a) introducing a sample into a treatment chamber;
b) removing, particularly by suction/evaporating the suspension medium and/or by filtering the sample in the treatment chamber;
c) determining the mass of the dry sample in the treatment chamber;
d) supplying heat energy to the sample in the treatment chamber until the sample is ashed;
e) determining the mass of the ashed sample residue in the treatment chamber.

2. Process according to claim 1, characterised in that the mass of the sample in the treatment chamber is determined before step b).

3. Process according to claim 1, characterised in that after step e) heat and oxygen is supplied to the ash until it is burnt out in the treatment chamber.

4. Process according to claim 3, characterised in that the mass of the burnt out sample residue is preferably determined in the treatment chamber.

5. Process according to claim 1, characterised in that a flocculent is supplied to the sample before removing the suspension medium.

6. Process according to claim 1, characterised in that at least at pre-determined time intervals a gas flow is supplied right through the sample.

7. Device according to claim 6, characterised in that the temperature of the gas flow is measured after flowing through the sample.

8. Process according to claim 4, characterised in that a curve of temperature and/or weight of the sample is recorded over time.

9. Process according to claim 8, characterised in that extreme values in the curve are obtained for determining certain processing states of the sample.

10. Process according to claim 1, characterised in that a drying state of the sample is ascertained, when a change in the mass of the sample in step b) falls short of a given value over time.

11. Process according to claim 7, characterised in that a drying state of the sample is ascertained, when a time curve of a temperature increase in the gas flow exceeds a pre-determined value.

12. Process according to claim 1, characterised in that a suction differential pressure, particularly suction underpressure and a drying state dependent on its time curve is ascertained when drawing off the suspension medium.

13. Process according to claim 1, characterised in that a drying state of the sample is ascertained by irradiating the sample with microwave radiation and ascertaining the absorption of the microwave rays, whereby irradiating the sample with microwave radiation is applied during drying preferably at least partially to heat up and/or evaporate the suspension medium.

14. Process according to claim 1, characterised in that in step d) the completion of the ashing of the sample is ascertained by essentially continuously observing the sample and/or by ashing resulting solids, particularly by CO₂, H₂O or smoke measurement or measurement of light emission or colour, or a combination thereof.

15. Process according to claim 8, characterised in that values that are not measured can be extrapolated from the curve.

16. Device for determining an organic and inorganic fraction of a sample comprising a suspension medium, particularly water, and solids particularly to carry out a process according to claim 1 with a preferably sealed treatment chamber, comprising
a sampling device;
a filter unit, which is constructed and attached to the sampling device such that the suspension medium can be removed from the sample through the filter unit;
a device to create a differential pressure, particularly a suction device to remove the suspension medium;
a heat energy source to supply heat energy to the sample;
a temperature measuring unit to record the temperature of the sample;
a weighing unit to weigh the sample;
and with a control and data evaluation unit to control the heat energy source and to record and store output signals from the temperature measuring unit and the weighing unit.

17. Device according to claim 16, characterised in that the filter unit comprises a filter suitable for combustion temperatures, preferably a glass fibre filter or a perforated plate filter that preferably can be replaced.

18. Device according to claim 16, characterised in that the heat energy source comprises a source for creating microwaves, e.g. within the range 2.455 GHz.

19. Device according to claim 16, characterised in that the heat energy source comprises a source for creating infrared radiation.

20. Device according to claim 16, characterised in that a sample supplying unit for particularly continuous or dropwise supply of a sample to a filter unit over a specific period of time.

21. Device according to claim 16, characterised in that a gas conditioning unit is provided to supply gas, particularly air, to the treatment chamber, whereby the gas conditioning unit comprises preferably units for conditioning, particularly heating and/or drying the supplied gas, and/or changing the gas e.g. from air to nitrogen.

22. Device according to claim 16, characterised in that the control and data evaluation unit comprises storage units and is constructed such that the weight of the sampling device is ascertained and recorded
in the wet state to ascertain the total sample mass; and/or
in the dry state to ascertain the sample content of dry mass; and/or
after ashing, preferably at a maximum of approximately 580° C., to ascertain the inorganic and organic fraction
after burning out, preferably at over 580° C., to ascertain the calcium carbonate fraction.

23. Device according to claim 16, characterised by heaters to introduce into the sample, which are constructed such that they can be heated up by the heat energy source.

24. Device according to claim 16, characterised by a flocculant to introduce into the sample and disperse it when removing the suspension medium.

25. Device according to claim 16, characterised by gas removal units, which are constructed and attached such that the sample can essentially have a continuous throughflow of gas.

26. Device according to claim 25, characterised in that the gas removal units comprise a unit creating differential pressure, particularly a vacuum pump.

27. Device according to claim 25, characterised in that the gas removal units comprise a temperature measuring unit to create a temperature—measuring signal that corresponds to the temperature of the gas flowing through the sample.

28. Device according to claim 16, characterised in that the control and data evaluation unit is constructed such that the stored output signals of temperature and weight can be evaluated using particular values, particularly extreme values from a curve of temperature and/or weight over time.

29. Use of a device according to claim 1 or of a process according to claim 14 to determine pigment and fibre fractions in a suspension for paper manufacture.

30. Use of a process according to claim 1 or of a process according to claim 14 to determine an organic and inorganic fraction in wastewater and slurries containing solid matter, particularly to minimise the addition of flocculants and/or to calculate waste disposal values.

31. Use of a device according to claim 1 to determine a pigment fraction in a paper coating.
